(19) United States
(12) Patent Application Publication
(10) Pub. No.: US 2002/0102230 A1
(43) Pub. Date: Aug. 1, 2002

(54) Method for inactivating pathogens

(76) Inventors: Tianxin Wang, Columbia, MD (US); Shazhou Zou, Glenwood, MD (US); Victor W. Wang, Herndon, VA (US)

Correspondence Address: BIRCH STEWART KOLASCH & BIRCH, PO BOX 747, FALLS CHURCH, VA, 22040-0747, US

(21) Appl. No.: 10/002,196

(22) Filed: Dec. 5, 2001

Related U.S. Application Data
(60) Provisional application No. 60/251,232, filed on Dec. 5, 2000.

(51) Int. Cl. A61K 31/74; A61K 31/795

(52) U.S. Cl. 424/78.27; 424/78.35

(57) ABSTRACT

A method for inactivating pathogens is provided. This method generally comprises the application of an effective amount of inhibitory compounds, preferably as a topical formulation, either alone or in combination with a pharmaceutically acceptable carrier or diluent. The inhibitory compounds include a copolymer of maleic acid and styrenesulfonic acid, polyvinyl phthalate sulphate, and their salts. The method can be used for preventing transmission and infection of sexual transmitted diseases, for treating and preventing bacterial vaginitis, and as a contraceptive method. These contraceptives generally have fewer side effects than conventional vaginal contraceptives (e.g., Nonoxynol-9). For example, the compounds useful in the methods of the invention not only are not toxic to natural and beneficial flora and, thus, do not upset the local microbiological balance, but also help maintain a low pH in the vaginal environment.

The present application claims priority under 35 U.S.C. §119(e) to U.S. Provisional Application No. 60/251,232, filed on Dec. 5, 2000, which provisional application is hereby incorporated by reference in its entirety.

### BACKGROUND OF THE INVENTION

1. Field of the Invention

The present invention is directed to compounds that inactivate pathogens, i.e., which possess anti-viral and anti-microbial activity. The compounds may therefore be used as a therapeutic agent for treating and preventing bacterial infections, including bacterial vaginosis or vaginitis, and preventing sexually transmitted diseases such as chlamydia trachomatis, genital herpes from herpes simplex viruses, acquired immunodeficiency syndrome from human immunodeficiency viruses, syphilis and gonorrhea.

2. Background Information

In the United States, an estimated 15.3 million new cases of sexually transmitted diseases (STDs) occur each year, one-quarter of them among teenagers. Of the top 11 reportable diseases in the United States in 1996, five were transmitted sexually, based on the report from Centers for Disease Control and Prevention. In the United States in 1994, approximately $10 billion was spent on major STDs (other than acquired immunodeficiency syndrome (AIDS) caused by human immunodeficiency viruses (HIV)). This figure rises to approximately $17 billion when cases of HIV infections are included (source: Committee on Prevention and Control of Sexually Transmitted Diseases). Worldwide, an estimated 333 million new cases of four types of STDs (i.e., gonorrhea, chlamydial infection, syphilis and trichomoniasis) occurred in 1997. Studies indicate that AIDS and other sexually transmitted diseases have reached an epidemic proportion. At the current rate, one in every four adults will be infected with a STD in his/her life. People with STDs are at an increased risk for HIV/AIDS infection. In Africa, HIV/AIDS infection has become a life and death issue not only for individuals, but also for entire nations. Battling STDS, such as HIV/AIDS, has become a global priority.

About 90% of all HIV infections occur during heterosexual intercourse. Condoms are still the single most effective measure to prevent STD infections, including HIV, for these circumstances. However, until the economical and educational levels of the general population reach a relatively high level, and women have more control over their sexual activities, the consistent uses of condoms during sexual intercourse will continue to prove to be unattainable. Still, even consistent use of condoms does not provide 100% protection against infection of a STD.

Some viral STDs are very prevalent, yet remain incurable. For example, about one in every five people in the United States over the age of 12, which is approximately 45 million individuals, are infected with herpes simplex virus (HSV), which is the virus that causes genital herpes. There is also an economic drawback in treating a STD. For example, the costs associated with the treatment of genital herpes totaled approximately $237 million in 1994. There is also evidence that infection by a STD virus is associated with more than 80 percent of cases of invasive cervical cancer.

Bacterial vaginitis is the major gynecological disease threatening women's health. Due to limited medical resources, the inconvenience of making a hospital visit, and a lack of medical knowledge, many women having bacterial vaginitis remain untreated. A microbicide that is useful in preventing and treating bacterial vaginitis would have great significance to women's health.

Nonoxynol-9 is a nonionic detergent with strong surfactant properties and has some potency in killing STD pathogens, including the HIV virus, and stopping STD infections. Nonoxynol-9 is a potent cytotoxic agent, which tends to nonspecifically disrupt cell membranes. These properties, however, give rise to some very significant disadvantages. Because Nonoxynol-9 is strongly cytotoxic, it can injure vaginal/cervical epithelial and other cells at concentrations as low as about 0.0005 percent. Clinical studies have confirmed epithelial disruption of the vagina and cervix. As another drawback, Nonoxynol-9 also disrupts the normal vaginal flora, which provides a protective mechanism, perhaps by maintaining a low pH, to guard against the invasion of pathogenic microbes. Nonoxynol-9 may also partially dissolve or remove the protective glycoprotein coating in the vagina. The cytotoxic, flora-disruptive, and glycoprotein-removal effects of Nonoxynol-9 can lead to vaginal damage or injury, including lesions. Some women are especially sensitive to Nonoxynol-9 and manifest these undesirable effects with even occasional use thereof. Because of the disruption of these protective mechanisms due to uses of Nonoxynol-9, such uses can actually increase the risks of STD infection, since the breakdown of the protective mechanisms, and especially the occurrence of lesions, provides STD-causing organisms with an easier pathway into the cells. Thus, any anti-STD activity of the contraceptive may be reduced or even lost (i.e., overwhelmed) by the increased risk of infection due to physical damage from the contraceptive. Even if such a contraceptive method provided some degree of STD protection, it would, of course, mainly be directed at heterosexual relationships in which pregnancy is not desired and not to offer protection against STDs.

Acid buffers also have been used for a long time to treat vaginal infections and disorders, as well as a spermicidal reagent. They also showed an effect in preventing sexually transmitted diseases. However, the potency of acid buffers is limited.

### SUMMARY OF THE INVENTION

In searching for effective antiviral compounds, which could be applied topically to decrease the frequency of sexual transmitted diseases, the applicants have discovered the present invention. The present invention involves the administration of a copolymer of maleic acid and styrenesulfonic acid, or a polymer of polyvinyl phthalate sulphate, and salts thereof, for treating and preventing infections, bacterial vaginitis, and preventing sexually transmitted diseases and pregnancy.

This invention relates to using inhibitory compounds to inactivate pathogens, where the pathogens can cause sexually transmitted diseases and bacterial vaginitis. These inhibitory compounds include (i) copolymers of maleic acid and styrenesulfonic acid, and (ii) polymers of polyvinyl phthalate sulphate, and salts thereof.

The present invention in another aspect is concerned with providing a novel polyvinyl phthalate sulphate class of polymers, and salts thereof.

### DETAILED DESCRIPTION

The following detailed description is provided as an aid to those desiring to practice the invention disclosed herein, it is not, however, to be construed as limiting to the instant invention as claimed, since those of ordinary skill in the art will readily understand that variations can be made in the procedures, methods, ingredients, ratios, and compositions disclosed herein, without departing from the spirit or scope of the instant invention. As such the present invention is only limited by the scope of the claims appended hereto and the equivalents encompassed thereby.

New methods have been invented involving two classes of polymers that are effective in inactivating pathogens. These two classes of polymers have been tested and show potent activity against pathogens of sexually transmitted diseases. The first class of polymers encompasses copolymers of maleic acid and styrenesulfonic acid. The second class of polymers encompasses polymers of polyvinyl phthalate sulphate, which can be mixed esters comprising phthalate and sulphate functional groups on a polyvinyl backbone, and which can be produced as an esterification product of polyvinyl alcohol by phthalic anhydride and sulfuric chloride. Each of these classes of compounds has a high density of acid functional groups, which groups make the compounds capable of acting as acid buffers. The acid buffer capability of the two classes of polymers makes them useful in inactivating pathogens, since most pathogens cannot survive under low pH acidic conditions. The acid buffer capacity of the polymers (and copolymers) and their anion property also lead to spermicidal activity, which also allows them to be utilized in methods of preventing pregnancy.

However, these compounds also possess other mechanisms to inactivate pathogens besides their acid buffer capacity. Acid buffer capacity requires that high concentrations of compounds to be applied, but in our tests, these compounds are highly active against pathogens in much lower concentrations than those required of an acid buffer. Therefore, these compounds inactivate pathogens via multiple actions. They showed high activity in virus fusion and attachment assay, suggesting one of their modes of action is acting as virus entry blockers, which prevent a virus from entering cells, and therefore prevent infections. These compounds could inhibit key receptors required for pathogen infection, and therefore inactivate pathogens.

The active ingredient of copolymers and polymers of the present invention, as well as suitable methods for the preparation thereof, are described more fully below.

For copolymers of maleic acid and styrenesulfonic acid that are useful in the present invention, the molecular weight ratio of the maleic acid to the styrenesulfonic acid can be varied freely in almost any amount (e.g., molecular weight ratios are effective at from 9:1 to 1:9; 7:3 to 3:7; and at about 1:1). Preferably, the molecular weight ratio of maleic acid to styrenesulfonic acid is about 1:1.

For polyvinyl phthalate sulphate, the molecular weight ratio of phthalate to sulphate can be varied freely in almost any amount as well (e.g., molecular weight ratios are effective from 9:1 to 1:9; 7:3 to 3:7; and about 1:1). The preferred molecular weight ratio is about 1:1.

The copolymers of maleic acid and styrenesulfonic acid can be made by well-known methods employing copolymerization of maleic acid with sulfonated styrene (e.g., Kobashi et al. U.S. Pat. No. 4,009,138), or by hydrolysis of a copolymer of maleic anhydrate and styrenesulfonic acid. The synthesis of copolymers of maleic anhydrate and styrenesulfonic acid is described by Bauman et al. (U.S. Pat. No. 2,835,655).

Accordingly, a suitable and exemplary method of preparing a copolymer of maleic acid and styrenesulfonic acid (molecular weight ratio of 1:1), is as follows: 2.1 g styrenesulfonic acid sodium salt and 1.2 g maleic acid is dissolved in 30 mL water; 0.04 g potassium peroxodisulfate is added. The mixture is heated at 95° C. for 5 hours. The solution is dried and is then washed with 100 mL acetone twice to give a white powder.

The above exemplary method possesses an advantage over the method taught by Bauman et al. (U.S. Pat. No. 2,835,655), in that there is no need to use a toxic organic solvent.

Similarly, a suitable and exemplary method for preparing a polymer of polyvinyl phthalate sulphate (molecular weight ratio of 1:1) is as follows: 4.4 g PVA (poly vinyl alcohol) is dissolved in 50 ml DMF at 100° C., and then 7.4 g phthalic anhydride is added. The mixture is stirred at 100° C. for 10 hours. The mixture is then cooled to 0° C. and 15 g chlorosulfonic acid is slowly added. The mixture is then kept under room temperature for 10 hours. Then 600 mL acetone is added to the mixture, and the resulting precipitate is collected and is dissolved in 100 ml water. The solution is extracted with 300 mL dichloromethane, and the water layer is lyophilized to give a final product in the form of white powder.

The above described instant classes of polymers have shown potent activity against HIV-1, HIV-2 and HSV-1 and HSV-2 (herpes simplex viruses). Although they do not possess such an effect on already infected cells in our tests, the polymers can nonetheless be advantageously used to protect healthy cells from being infected by a HIV virus. Moreover, their capability to inactivate pathogens indicates that the polymers possess suitable properties for applications in preventing sexually transmitted diseases and treating bacterial vaginitis.

One important requirement for topically applied drugs in preventing sexually transmitted diseases is that they must be safe for the local vaginal environment, since they must be used frequently as prevention tools. The drugs should not cause any irritation to human membrane. Nonoxynol-9 lacks safety because it causes severe irritation to human vaginal membrane and lesions. Our compounds show high anti-microbial activity in our cell based assays and very high safety index indicated by the very high CC₅₀ to normal cells (drug concentration causing 50% cell death). This high safety index is crucial for effective application against infection caused by pathogens since the failure of Nonoxynol-9 is due to its low safety index (low CC₅₀) to normal cells.

The safety of the instant polymers was tested by applying them to rat vaginas. No signs of irritation were shown at concentrations up to 15% after being continually applied for 7 days. The compounds also did not inhibit the growth of beneficial vaginal Lactobacillus. Lactobacillus are beneficial vaginal bacteria, because they maintain normal vaginal acidity and inhibit the growth of other pathogens that may cause infections. Therefore, both compounds could be used as effective topically applied compounds to prevent sexually transmitted diseases.

The present invention is directed to several methods of utilizing these polymers. The polymers are preferably administered as part of a pharmaceutical composition, provided that the active ingredient is present in the composition in an effective amount. The effective amount range of the polymer (or copolymer) as an active ingredient typically varies from about 0.5 mg to 5 g per dose, with the preferred dosage range of the active ingredient being from about 250 mg to 2.5 g per dose.

Typically, the pharmaceutical compositions of the invention comprise a pharmaceutically acceptable carrier or diluent in combination with the active ingredient of the invention (i.e., the copolymer of maleic acid and styrenesulfonic acid, and/or the polymer of polyvinyl phthalate sulphate, and salts thereof). Such pharmaceutically acceptable carriers or diluents should not be detrimental to the ability of the active ingredient to produce or provide its intended effect. Suitable pharmaceutically acceptable carriers and diluents are generally readily known to those skilled in the art. Exemplary of suitable carriers and diluents are pharmaceutical excipients for formulation set forth in the United States Pharmacopeia-National Formulary (USP-NF), the most current available edition of which (USP 25 -NF 20) is herein incorporated by reference in its entirety.

Such pharmaceutical compositions are preferably in a form that can be applied easily, and quickly, while offering a substantial amount of coverage/protection to the body part(s) or body portion(s) to which they are applied, to thereby ensure that the desired effects from active ingredients of the present invention are produced.

Preferably, the pharmaceutical compositions of the present invention include, but are not limited to, topically applied compositions, which can be applied to portion(s) or part(s) of the body of an individual that will be (or have recently been) involved in sexual activity or sexual contact with another individual (e.g., vagina, labia, clitoris, vulva, breast, penis, scrotum sac, anus, hands, fingers, lips, etc.), with the administration of the compositions taking place either prior to, and/or if by necessity or if otherwise required, immediately or closely after such sexual activity or sexual contact (e.g., within 1-30 minutes, or within several minutes up to 1-8 hours after sexual contact). Most preferably, the compounds or compositions of the invention are administered prior to the occurrence of sexual contact or sexual activity to help ensure their effectiveness and efficacy in the inventive methods.

The term sexual contact or sexual activity, as used herein is meant to include, but is not limited to, physical sexual contact between individuals that involves the genitalia of at least one person, and which type of sexual contact is normally associated with or involves a transmission or release of a bodily fluid (e.g., vaginal intercourse, anal intercourse, fellatio, and self or mutual stimulation (masturbation), and the like).

The use of such compositions of the invention in combination with other means to prevent the transmission of STDs (or to prevent pregnancy) is also contemplated herein, such as the use thereof in combination with the use of prophylactics or condoms.

The compositions of the present invention, due to the presence of the active ingredient polymers and/or copolymers therein, are effective in preventing the occurrence of sexually transmitted diseases such as those that are, or that are caused by, chlamydia trachomatis, herpes simplex virus, human immunodeficiency virus, syphilis, gonorrhea, and papilloma virus, or the like.

Exemplary of suitable pharmaceutical compositions of the present invention that can be used in the various methods described herein are those that can be applied quickly and easily to body portion(s) or body part(s), and are most preferably topically applied compositions, including those in the forms of liquids, sprays, aerosols, balms, ointments, gels, oils, creams, lotions, suspensions, suppositories, emulsions, wet-wipes, and the like. As indicated previously, such compositions should be capable of allowing one to administer an active ingredient polymer (or copolymer) of the present invention, in an effective amount of about 0.5 mg to 5 g per dose, preferably about 250 mg to 2.5 g per dose, without inhibiting the active ingredient's ability to produce or carry out its intended effect according to the method(s) of the instant invention.

The various methods of the present invention are now described more fully and particularly.

A first method of the invention pertains to a method of inactivating pathogens in an individual in need thereof by administering to the individual an effective amount of an active ingredient compound or pharmaceutical composition of the invention. The method typically comprises administering a pharmaceutical composition comprising an effective pathogen inactivating amount of (i) a copolymer of maleic acid and styrenesulfonic acid, or a salt thereof, or (ii) polyvinyl phthalate sulphate, or a salt thereof, wherein the polyvinyl phthalate sulphate can be a mixed ester comprising phthalate and sulphate functional groups on a polyvinyl backbone; to thereby inactivate the pathogens. The pharmaceutical composition preferably further comprises a pharmaceutically acceptable carrier or a diluent as described hereinabove, with the effective pathogen inactivating amount of the active ingredient compound being from about 0.5 mg to 5 g per dose, and preferably from about 250 mg to 2.5 g per dose. In the method, the pharmaceutical composition can be administered as needed, and is preferably administered from one to three times a day.

A second method of the invention is directed to preventing a transmission of, or infection by, sexually transmitted diseases (STDs) in an individual in need thereof. This method comprises administering an active ingredient compound or pharmaceutical composition of the present invention to a portion(s) or part(s) of the body of an individual that will be, or that have recently been, engaged in a sexual activity or a sexual contact, wherein the administration preferably occurs prior to said sexual activity or contact, or if necessary or otherwise required, immediately or closely after such sexual activity or contact (e.g., within 1-30 minutes after such sexual activity or contact, or within several minutes up to 1-8 hrs after such sexual activity or sexual contact). In the instance where a pharmaceutical composition of the invention is administered, the pharmaceutical composition comprises an effective amount of (i) a copolymer of maleic acid and styrenesulfonic acid, or a salt thereof, or (ii) polyvinyl phthalate sulphate, or a salt thereof, wherein the polyvinyl phthalate sulphate can be a mixed ester comprising phthalate and sulphate functional groups on a polyvinyl backbone; to thereby prevent the transmission of, or infection by, STDs as a result of said sexual activity or sexual contact. The effective amount of the active ingredient compound (i.e., the polymer, copolymer, or salt thereof) in the pharmaceutical composition is about 0.5 mg to 5 g per dose, and preferably about 250 mg to 2.5 g per dose.

A third method of the invention pertains to treating and/or preventing bacterial vaginitis in a female individual in need thereof, where the method comprises administering to the vaginal area of the female individual an effective amount of an active ingredient compound of the invention, or a pharmaceutical composition comprising an effective amount of (i) a copolymer of maleic acid and styrenesulfonic acid, or a salt thereof, or (ii) polyvinyl phthalate sulphate, or a salt thereof, wherein the polyvinyl phthalate sulphate can be a mixed ester comprising phthalate and sulphate functional groups on a polyvinyl backbone; to thereby treat and/or prevent bacterial vaginitis. The effective amount of the active ingredient polymer, copolymer or salt thereof may be 0.5 mg to 5 g per dose, and is preferably about 250 mg to 2.5 g per dose. In the method, the pharmaceutical composition can be administered as needed, and is preferably administered from one to three times a day.

A fourth method of the instant invention is a contraceptive method for preventing pregnancy in a female individual in need thereof. This method comprises administering to the vaginal area of the female individual an effective amount of an active ingredient compound of the invention, or a pharmaceutical composition comprising an effective pregnancy preventing amount of (i) a copolymer of maleic acid and styrenesulfonic acid, or a salt thereof, or (ii) polyvinyl phthalate sulphate, or a salt thereof, wherein the polyvinyl phthalate sulphate can be a mixed ester comprising phthalate and sulphate functional groups on a polyvinyl backbone; to thereby prevent pregnancy in the female individual. The effective pregnancy preventing (contraceptive) amount of the active ingredient is from about 0.5 mg to 5 g per dose, and is preferably about 250 mg to 2.5 g per dose.

In the contraceptive method of the present invention, the composition is most preferably administered to the vaginal area of the female individual prior to vaginal intercourse in order to best insure the prevention of pregnancy in the female individual, but if necessary or otherwise required, it can be applied to the vaginal area of the female individual immediately after, or closely after vaginal intercourse (e.g., within 1-30 minutes after vaginal intercourse, or within from several minutes up to 1-8 hrs after vaginal intercourse). When the active ingredient containing pharmaceutical composition is applied or administered after vaginal intercourse, it is preferably applied immediately, e.g., within 1-30 minutes after completion of the act of intercourse, to help ensure the prevention of pregnancy in the female individual according to the methods of the present invention.

To further aid those desiring to practice the methods of the present invention, the following demonstrative Examples are provided. These examples are intended to illustrate the invention and the advantageous properties possessed thereby. Their disclosure herein does not limit the scope of the appended claims in any way, or the scope of equivalents encompassed thereby.

### EXAMPLES

Against HIV-1

Both compounds were tested in the standard suite of topical microbicide evaluation assays supplied by the National Institute of Allergy and Infectious Disease. Both CD4-independent HIV transmission inhibition assay and the CD4-dependent HIV transmission inhibition assay were carried out. The procedures are described as follow.

CD4-Independent HIV Transmission Inhibition Assay:

ME180 cells, a CD4 negative, X4 positive cervical epithelial cell line is maintained in RPMI 1640 supplemented with 10% fetal bovine serum, glutamine and antibiotics. Twenty-four hours prior to the assay, ME180 cells are trypsonized, washed and seeded in 96-well flat bottom microtiter plates at a density of 2×10³ cells per well. On the day of the assay, H9 cells chronically infected with the SK1 clinical isolate of HIV-1 (H9-SK1) are treated with freshly made mitomycin C (200 μg/ml) for 60 minutes at 37° C. This concentration of mitomycin C is sufficient to result in cell death, but allows viral transmission to occur. After mitomycin C treatment, the H9-SK1 cells are washed three times with tissue culture media. Test compounds are added to the ME180 monolayer followed by 2×10⁴ H9-SK1 cells. The ME180 cells are co-cultured with H9-SK-1 cells and test material for 4 h, and the H9-SK1 cells are removed by washing the ME180 monolayer three times with PBS. At 24 and 48 h post assay initiation the wells are again washed three times with PBS to ensure removal of the H9-SK1 cells, and culture continued in test material free media. At six days post-co-cultivation, supernatants are collected and evaluated for p24 antigen expression by ELISA. Cell viability is assessed by XTT dye reduction. Compounds that are judged as active in the first test are retested with or without the addition of mucin. Testing in the presence of mucin is carried out by addition of 200 μg/ml of porcine mucin (Sigma Chemical Co., St Louis, Mo.) to the transmission reactions. Transmission intervals and washing without replacement of mucin or compound are carried as described above. All determinations are performed in triplicate with serial ½ Log₁₀ dilution of the test materials.

CD4-Dependent HIV Transmission Inhibition Assay:

The CD4-dependent HIV transmission inhibition assay is carried out essentially as described for the CD4-independent transmission assay except for the use of the CD4 positive GHOST (3) X4/R5 cell line. This cell line is derived from the HOS (human osteosarcoma) cell line that is negative for HIV coreceptor and CD4 expression. The cell line is engineered to express T4 (CD4), R5 and X4 via non-selectable retroviral vectors and an HIV-2 LTR hGFP construct with a hygromycin selectable marker. The cell lines are handled and cultured as described above for the CD4-independent HIV inhibition assay, with the exception that 2.5×10⁴ GHOST (3) X4/R5 and 5×10² mitomycin C treated H9/SK-1 cells are used in the assay. Addition of compounds, mitomycin C treatment and post-transmission washing to remove the H9/SK-1 cells are performed as described above to allow comparability of the two antiviral assays. Virus replication is assessed at 24 h post infection, following 3 washes, by measurement of cell-associated p24 by ELISA to ensure a single round of infection in the presence of CD4. Compound toxicity and cell viability are assessed by XTT dye reduction. Compounds that are judged as active in the first test are retested with or without the addition of mucin. Testing in the presence of mucin is carried out by addition of 200 μg/ml of porcine mucin (Sigma Chemical Co., St Louis, Mo.) in tissue culture medium to the transmission reactions. Transmission intervals and washing without replacement of mucin or compound are carried as described above. All determinations are performed in triplicate with serial ½ Log₁₀ dilution of the test materials.

Both compounds are highly active in the CD4-independent HIV transmission inhibition assay and the CD4-dependent HIV transmission inhibition assay (Table 1).

TABLE 1
Activities against HIV in the CD4-independent HIV
transmission inhibition assay and the CD4-dependent HIV
transmission inhibition assay with or without mucin. Mucin
was used to mimic the complex environment of the vagina.
IC50 is the drug concentration inhibiting 50% of viral
infection. TC50 is the drug concentration showing toxicity
to 50% normal cells. TI is the therapeutic index, which
equals to TC50/IC50.

| | | CD4-Independent | | | CD4-Dependent | | |
|---|---|---|---|---|---|---|---|
| | | Transmission | | | Transmission | | |
| | | Assay (μg/ml) | | | Assay (μg/ml) | | |
| Compound | Mucin | IC₅₀ | TC₅₀ | TI | IC₅₀ | TC₅₀ | TI |
| Copolymer of | − | 0.93 | >100 | >107 | 1.2 | >500 | >416 |
| maleic acid and | + | 0.86 | >100 | >116 | 0.99 | >250 | >252 |
| styrenesulfonic | | | | | | | |
| acid. | | | | | | | |
| Polyvinyl | − | 1.4 | >50 | >35 | 1.8 | >100 | >56 |
| phthalate | + | 0.9 | >50 | >59 | 1.7 | >150 | >59 |
| sulphate | | | | | | | |

Both compounds are also active in preventing HIV virus attachment and fusion, suggesting they act as a viral entry inhibitor (Table 2). The detailed procedures are described below.

Virus Attachment Assay

This assay is designed to detect compounds that interact with the cell and block virus attachment, and/or compounds that interact with the forming attachment/fusion complex. The attachment assay is performed with HeLa CD4 LTR β-gal cells. HeLa CD4 LTR β-gal cells are routinely cultured with the required selection antibiotics. Twenty-four hours prior to initiation of the assay the cells are trypsinized, counted, and 1×10⁴ cells placed in a 0.2 cm well in media without selection antibiotics. At 24 h media is removed and compound in media placed on the cells and incubated for 15 min at 37° C. A known titer of the IIIB strain of HIV is then added to the wells and the incubation continued for 1 h. At the end of the incubation, the wells are washed 3 times with media and the culture continued for 40 to 48 h. At termination of the assay, media is removed and β-galactosidase enzyme expression determined by chemiluminescence per manufacturer instructions (Tropix Gal-screen™, Bedford Mass.) by a single step chemiluminescent method using a single solution to lyse the cells and detect β-galactosidase enzyme activity. Compound toxicity is monitored on a sister plate by XTT dye reduction. All determinations are performed in triplicate with serial ½ Log₁₀ dilution of the test materials. The virus adsorption interval of 1 h is sufficiently short that AZT, which requires phosphorylation to its active tri-phosphate form (AZT-TTP), is not active in this assay.

Fusion Assay

The fusion assay assesses the ability of compounds to block cell-to-cell fusion mediated by HIV-1 Env and CD4 expressed on separate cells. This assay is sensitive to inhibitors of both the gp120/CD4 interaction and inhibitors of the X4 coreceptor. First, 5×10³ HeLa CD4 LTR β-gal cells are placed in microtiter wells and incubated overnight. The following day the media is removed and the HeLa CD4 LTR β-gal cells are incubated for 1 h at 37° C. in fresh media with test compound. Following the incubation 5×10³ HL2/3 cells are added and the incubation continued for 40 to 48 h. At 40 to 48 h β-galactosidase enzyme expression is detected by chemiluminescence (Tropix Gal-screen™, Tropix, Bedford, Mass.). Compound toxicity is monitored on a sister plate using XTT dye reduction. All determinations are performed in triplicate with serial ½ Log₁₀ dilution of the test materials.

TABLE 2
Inhibitions of HIV Virus Attachment and Fusion.

| | Attachment Assay | | | Fusion Assay | | | |
|---|---|---|---|---|---|---|---|
| | (μg/ml) | | | (μg/ml) | | | |
| Compound | IC₅₀ | TC₅₀ | TI | IC₅₀ | TC₅₀ | TI | Comments |
| Chicago Sky | 0.32 | >10 | >31 | 0.5 | >10 | >19 | Control |
| Blue | | | | | | | compound |
| Copolymer | 0.04 | >1 | >25 | 1.1 | >50 | >45 | Inhibited |
| of maleic | | | | | | | Virus |
| acid and | | | | | | | Attachment |
| styrene- | | | | | | | and Fusion |
| sulfonic | | | | | | | |
| acid | | | | | | | |
| Polyvinyl | 0.22 | >50 | >227 | 2.2 | >50 | >22 | Inhibited |
| phthalate | | | | | | | Virus |
| sulphate | | | | | | | Attachment |
| | | | | | | | and Fusion |

Both compounds were also evaluated for their activity against cytopathic effects of the infection of CEM-SS cells by cell free virus HIV-1 (RF) IC₅₀ and CC₅₀ (the drug concentration showing toxicity to 50% normal cells) determined from the cytopathic effects is described in the following assay. CEM-SS cells (obtained from the AIDS Research and Reference Reagent Repository, Bethesda, Md.) are passaged in T-75 flasks in tissue culture media (RP 1640 medium (no phenol red) with 10% Fetal Bovine Serum (heat inactivated), 2 mM L-glutamine, 100 U/mL penicillin, 100 μg/mL streptomycin, and 10 μg/mL gentamycin). On the day preceding the assay, the cells are split 1:2. On the day of assay the cells are collected by centrifugation, washed twice with tissue culture medium and resuspended in fresh tissue culture medium. Total cell and viability counting is performed using a hemacytometer. Cell viability prior to the assay is determined by Trypan Blue dye exclusion and must exceed 95%. A pretitered aliquot of HIV-1 RF (AIDS Research and Reference Reagent Repository, Bethesda, Md.), CEM-SS cells and compound are placed into microtiter plates. Each plate contains cell control wells (cells only), virus control wells (cell plus virus), drug toxicity control wells (cells plus drugs only), drug calorimetric control wells (drugs only) as well as experimental wells (drug plus cells plus virus) . Cultures are incubated for 6 days at 37° C., 5% CO₂ and antiviral activity and compound toxicity determined by MTS staining. Activity is confirmed by both macroscopic and microscopic analysis of the assay. AZT is used as a control. Their activities are shown in Table 3. Both polyvinyl phthalate sulphate and copolymer of maleic acid and styrenesulfonic acid are highly active against HIV-1 virus infection.

TABLE 3
Activity against HIV-1 in CEM-SS cells.

| Compound | Ic₅₀ (μg/ml) | CC₅₀ (μg/ml) |
|---|---|---|
| AZT (μM) | 0.005 | >1 |
| Copolymer of maleic acid | 1.53 | >500 |
| and styrenesulfonic | | |
| acid. | | |
| Polyvinyl phthalate | 1.79 | >500 |
| sulphate | | |

Against HIV-2

Inhibition of the infection of CEM-SS cells by cell free virus HIV-2 (Rod) IC₅₀ and CC₅₀ determined from the cytopathic effects is described in the following assay. CEM-SS cells (obtained from the AIDS Research and Reference Reagent Repository, Bethesda, Md.) are passaged in T-75 flasks in tissue culture media (RP 1640 medium (no phenol red) with 10% Fetal Bovine Serum (heat inactivated), 2 mM L-glutamine, 100 U/mL penicillin, 100 μg/mL streptomycin, and 10 μg/mL gentamycin). On the day preceding the assay, the cells are split 1:2. On the day of assay the cells are collected by centrifugation, washed twice with tissue culture medium and resuspended in fresh tissue culture medium. Total cell and viability counting is performed using a hemacytometer. Cell viability prior to the assay is determined by Trypan Blue dye exclusion and must exceed 95%. A pretitered aliquot of HIV-2 Rod (AIDS Research and Reference Reagent Repository, Bethesda, Md.), CEM-SS cells and compound are placed into microtiter plates. Each plate contains cell control wells (cells only), virus control wells (cell plus virus), drug toxicity control wells (cells plus drugs only), drug calorimetric control wells (drugs only) as well as experimental wells (drug plus cells plus virus). Cultures are incubated for 6 days at 37° C., 5% CO₂ and antiviral activity and compound toxicity determined by MTS staining. Activity is confirmed by both macroscopic and microscopic analysis of the assay. Dextran sulfate (DS) is used as a control. Their activities are shown in Table 4. Copolymer of maleic acid and styrenesulfonic acid is highly active against HIV-2 virus infection.

TABLE 4
Activity of copolymer of maleic acid and
styrenesulfonic acid against HIV-2 virus.

| Compound | Ic₅₀ (μg/ml) | CC₅₀ (μg/ml) |
|---|---|---|
| DS | 0.27 | >10 |
| Copolymer of maleic acid | 0.37 | >1000 |
| and styrenesulfonic | | |
| acid. | | |

Against HSV-1

A virus induced cytopathic effects (CPE) inhibition assay procedure using Promega's cell titer aqueous one solution (MTS, metabolic dye similar to XTT) is employed to evaluate compounds for antiviral activity against herpes simplex virus type 1 (HSV-1) strain HF in Vero cells. Vero cells are pregrown in 96 well tissue culture plates using Dulbecco's modified eagle's media (DMEM) supplemented with 10% heat inactivated fetal bovine serum (FBS), L-glutamine, penicillin and streptomycin. Antiviral assays are designed to test six half log dilution of each compound in triplicate against the challenge virus in microtiter plate wells containing host cell monolayers. To each of the replicate cell cultures is added 50 uL of the test drug solution and 50 uL of virus suspension. Cell control containing medium alone, virus infected controls containing medium and virus, drug cytotoxicity controls containing medium and each drug concentration, reagent controls containing culture medium only (no cells), and drug calorimetric controls containing drug and medium (no cells) are run simultaneously with the test samples. The plates are incubated at 37° C. in a humidified atmosphere containing 5% CO₂ until maximum CPE is observed in the untreated virus control cultures (day 5). CPE inhibition is determined by the dye (MTS) uptake procedure. This method measures cell viability and is based on the reduction of the tetrazolium MTS by mitochondria enzymes of viable host cells to MTS formazon. The minimum inhibitory drug concentration which reduces the CPE by 50% (IC₅₀) and the minimum drug concentration which inhibits cell growth by 50% (CC₅₀) are calculated using regression analysis program for semilog curve fitting. Their activities are shown in Table 5. The inventive compound is highly active against HSV-1 virus.

TABLE 5
Activity of copolymer of maleic acid and
styrenesulfonic acid against HSV-1 virus.

| Compound | Ic₅₀ (μg/ml) | CC₅₀ (μg/ml) |
|---|---|---|
| Acyclovir | 2.7 μM | >10 |
| Copolymer of maleic acid | 3.2 | >1000 |
| and styrenesulfonic | | |
| acid | | |

Against HSV-2

Copolymer of maleic acid and styrenesulfonic acid was also evaluated for its activity against HSV-2 virus. Its IC₅₀ and CC₅₀ (Table 6) were determined from inhibition of the infection by HSV-2 virus in HFF cells, plaque reduction assay carried out by National Institute of Allergy and Infectious Disease. It is highly active against HSV-2 virus infection.

TABLE 6
Activity of copolymer of maleic acid and
styrenesulfonic acid against HSV-2 virus.

| Compound | Ic₅₀ (μg/ml) | CC₅₀ (μg/ml) |
|---|---|---|
| Copolymer of maleic acid | 0.41 | >100 |
| and styrenesulfonic | | |
| acid. | | |

When cells were already infected with the virus, the drug showed no activity to protect the cells from virus caused cell death. So it can not be used to treat virus infected cells. However, it showed high activity to protect cells from infection when drugs were added before cells were infected by the virus.

Acute Irritation Studies

This test evaluates the safety of the polymers as topically used microbicides.

A gel can be made by 10 parts (by weight) of active ingredient (copolymers of maleic acid and styrenesulfonic acid) dissolved in 88 parts water, and 2 parts carbopol 975 slowly added. The mixture is stirred for 1 hour in hot water bath. The pH value is adjusted to pH=4 using 1M NaOH aqueous solution, where the final product is a clear gel that could be applied topically.

An acute irritation study was carried out for the copolymer of maleic acid and styrenesulfonic acid. A total of 40 rats were divided into 4 groups. One group was used as a control, where the other three groups received gel containing 5%, 10% and 15% compound respectively for 7 days. The gel was applied topically to the vaginas of rats 0.2 ml each day, 7 days continuously. After 7 days administration, histopathology study indicated that no sign of irritation was observed for each rat. Therefore, the tested copolymers of maleic acid and styrenesulfonic acid showed potential for the application as a safe microbicide.

Effects on Beneficial Lactobacillus sp.

Lactobacillus are beneficial vaginal bacteria. Lactobacillus are important in maintaining normal vaginal acidity and therefore inhibit the growth of other pathogens which may cause infections. A good topical microbicide should not inhibit the growth of beneficial vaginal Lactobacillus. Thus, compounds of the instant invention (copolymer of maleic acid and styrenesulfonic acid, and polyvinyl phthalate sulphate) were tested for inhibition of beneficial Lactobacillus sp. growth.

The procedures were described as follow:

Lactobacillus crispatus and Lactobacillus jensenii were obtained from the American Type Tissue Culture Collection and grown in Lactobacilli MRS broth (Difco, Fisher Scientific, Pittsburgh, Pa.). This medium allows efficient growth of the Lactobacilli under anaerobic conditions. Bacillus stocks are produced and frozen in 15% glycerol at−80° C. for use in the sensitivity assay. To assess the effect of compounds on L. crispatus and L. jensenli growth, 10 ml of MRS media is inoculated with a stab from the glycerol bacterial stock. The culture is placed in a Gas Pak CO₂ bag and incubated for 24 h at 37° C. The next day the lactobacillus cultures are diluted to an OD of 0.06 at a wavelength of 670 nm. Compounds are diluted and placed into a 96 well flat bottomed plate and the Lactobacillus sp. was added. Commercially available Penicillin/Streptomycin solution at a high test concentration of 1.25 U/ml and 1.25 μg/ml, respectively, are used as the positive control. The plates are again incubated for 24 h at 37° C. in a Gas Pak CO₂ bag and bacterial growth determined by measurement of optical density at 490 nm in a molecular devices plate reader. All determinations are performed with 6½ log dilutions from a high-test concentration in triplicate.

Both L. crispatus IC₅₀ and L. jensaii IC₅₀ are greater than 500 μg/ml for the tested compounds of the invention in the inhibition of Lactobacillus sp. growth tests. The compounds do not substantially inhibit the growth of beneficial vaginal bacteria. Therefore, they are advantageous for application as a topical microbicide.

What is claimed is:

1. A method for inactivating pathogens in an individual in need thereof, said method comprising:
administering to the individual a pharmaceutical composition comprising an effective pathogen inactivating amount of a copolymer of maleic acid and styrenesulfonic acid, or a salt thereof, and a pharmaceutically acceptable carrier or diluent therefor.

2. The method according to claim 1, wherein the effective pathogen inactivating amount of the inhibitor compound is from 0.5 mg to 5 g per dose.

3. The method according to claim 1, wherein the pharmaceutical composition is administered topically.

4. A method for inactivating pathogens in an individual in need thereof, said method comprising:
administering to the individual a pharmaceutical composition comprising an effective pathogen inactivating amount of polyvinyl phthalate sulphate, or a salt thereof, and a pharmaceutically acceptable carrier or diluent therefor.

5. The method according to claim 4, wherein the polyvinyl phthalate sulphate is a mixed ester comprising phthalate and sulphate functional groups on a polyvinyl backbone.

6. The method according to claim 4, wherein the effective pathogen inactivating amount is 0.5 mg to 5 g per dose.

7. The method according to claim 4, wherein the pharmaceutical composition is administered topically.

8. A method for preventing transmission or infection of sexually transmitted diseases in an individual in need thereof, said method comprising:
administering a pharmaceutical composition to a portion or part of the body of said individual that is involved in a sexual activity or a sexual contact, said administration being prior to said sexual activity or sexual contact, or immediately thereafter, said pharmaceutical composition comprising an effective amount of a copolymer of maleic acid and styrenesulfonic acid, or a salt thereof, and a pharmaceutically acceptable carrier or diluent therefor; and
wherein said sexually transmitted disease is caused by, or is chlamydia trachomatis, herpes simplex virus, syphilis, gonorrhea or papilloma virus.

9. A method for preventing transmission or infection of sexually transmitted diseases in an individual in need thereof, said method comprising:
administering a pharmaceutical composition to a portion or part of an individual's body that is involved in a sexual activity or a sexual contact, said administration being prior to said sexual activity or sexual contact, or immediately thereafter, said pharmaceutical composition comprising an effective amount of a copolymer of maleic acid and styrenesulfonic acid, or a salt thereof, and a pharmaceutically acceptable carrier or diluent therefor; and
wherein said sexually transmitted disease is caused by human immunodeficiency virus (HIV).

10. The method according to claim 8 or 9, wherein the effective amount is 0.5 mg to 5 g.

11. The method according to claim 8 or 9, wherein the pharmaceutical composition is administered topically.

12. A method for preventing transmission or infection of sexually transmitted diseases in an individual in need thereof, said method comprising:
administering a pharmaceutical composition to a portion or part of an individual's body that is involved in a sexual activity or a sexual contact, said administration being prior to said sexual activity or sexual contact, or immediately thereafter, said pharmaceutical composition comprising an effective amount of a polyvinyl phthalate sulphate, or a salt thereof, and a pharmaceutically acceptable carrier or diluent therefor; and
wherein said sexually transmitted disease is caused by or is chlamydia trachomatis, herpes simplex virus, syphilis, gonorrhea or papilloma virus.

13. A method for preventing transmission or infection of sexually transmitted diseases in an individual in need thereof, said method comprising:
administering a pharmaceutical composition to a portion or part of an individual's body that is involved in a sexual activity or a sexual contact, said administration being prior to said sexual activity or sexual contact, or immediately thereafter, said pharmaceutical composition comprising an effective amount of a polyvinyl phthalate sulphate, or a salt thereof, and a pharmaceutically acceptable carrier or diluent therefor; and
wherein said sexually transmitted disease is caused by human immunodeficiency virus (HIV).

14. The method according to claim 12 or 13, wherein the effective amount is 0.5 mg to 5 g per dose.

15. The method according to claim 12 or 13, wherein the pharmaceutical composition is administered topically.

16. A method for treating and preventing bacterial vaginitis in a female individual in need thereof, said method comprising
administering to the vaginal area of said female individual, a pharmaceutical composition comprising an effective amount of a copolymer of maleic acid and styrenesulfonic acid, or a salt thereof, and a pharmaceutically acceptable carrier or diluent therefor.

17. The method according to claim 16, wherein the effective amount is 0.5 mg to 5 g per dose.

18. A method for treating and preventing bacterial vaginitis in a female individual in need thereof, said method comprising
administering to the vaginal area of said female individual, a pharmaceutical composition comprising an effective amount of a polyvinyl phthalate sulphate, or a salt thereof, and a pharmaceutically acceptable carrier or diluent therefor.

19. The method according to claim 18, wherein the effective amount is 0.5 mg to 5 g per dose.

20. A method for preventing pregnancy in a female individual in need thereof, said method comprising
administering to the vaginal area of said female individual a pharmaceutical composition comprising an effective amount of a copolymer of maleic acid and styrenesulfonic acid, or a salt thereof, and a pharmaceutically acceptable carrier or diluent therefor.

21. A method for preventing pregnancy in a female individual in need thereof, said method comprising
administering to the vaginal area of said female individual a pharmaceutical composition comprising an effective amount of a polyvinyl phthalate sulphate, or a salt thereof, and a pharmaceutically acceptable carrier or diluent therefor.

22. The method according claim 20 or 21, wherein the effective amount is 0.5 mg to 5 g per dose.

23. The method according to claim 20 or 21, wherein the effective amount is administered to the vaginal area of said female individual prior to vaginal intercourse or immediately thereafter.

24. A polyvinyl phthalate sulphate polymer, or a salt thereof.

25. A polyvinyl phthalate sulphate polymer, or a salt thereof, wherein said polymer or salt thereof is a mixed ester comprising phthalate and sulphate functional groups on a polyvinyl backbone.
